(19) United States
(12) Patent Application Publication
(10) Pub. No.: US 2011/0195130 A1
(43) Pub. Date: Aug. 11, 2011

(54) EDIBLE COMPOSITIONS

(76) Inventor: Sanford Siegal, Miami, FL (US)

(21) Appl. No.: 12/702,656

(22) Filed: Feb. 9, 2010

(51) Int. Cl. A61K 33/08; A61K 47/36; A61K 33/24; A61K 33/10

(52) U.S. Cl. 424/601; 514/777; 424/691; 424/686; 424/653; 424/687

(57) ABSTRACT

Disclosed are various embodiments of edible food compositions effective for neutralizing stomach acid. Further, the present invention is directed toward methods of preparing the edible composition and the methods for reducing stomach acidity by ingesting the edible composition. In one embodiment, the edible composition comprises sweetener, flour, fat, leavening agent, water, and an effective amount of acid neutralizing material. In one embodiment, the above ingredients are combined to form a heavy dough which is then rolled out, divided into sections, and baked, resulting in a cookie with effective antacid properties. Further disclosed are edible compositions for delivering calcium as a dietary supplement.

### TECHNICAL FIELD

The present invention relates generally to compositions and methods of preparing dietary and food products that are useful as dietary supplementation and for neutralizing stomach acid. More specifically, the present invention relates to compositions and methods that are intended to provide consumers with a better tasting alternative to traditional acid neutralizing delivery methods known in the art such as liquids, tablets, and pills.

### BACKGROUND OF THE INVENTION

Acid neutralizing agents are generally known in the art for temporarily relieving the symptoms of a wide variety of stomach ailments such as heartburn, acid reflux, and general indigestion. Generally speaking, the purpose of such agents is to perform a neutralization reaction, i.e. buffer gastric acid, raising the pH and thus reducing acidity in the stomach. As a point of reference, according to applicable U.S. federal regulations, a substance is deemed an effective antacid if the substance (whether a single antacid material, or a combination of known antacid materials) contains at least 5 mEq of acid neutralizing capacity as measured by the procedure provided in the United States Pharmacopeia 23/National Formulary 18. The United States Pharmacopoeia procedure states that a substance is an effective at neutralizing stomach acid at its lowest dose if, when added to 10 mL of 0.5 N HCL (5 mEq), produces a pH of 3.5 or greater after 10 minutes of stirring. Further, when considering a combination of effective acid neutralizing ingredients in a single dose, the regulations dictate that the following testing procedure should be followed to determine the percent contribution of each acid neutralizing ingredient: (1) place an accurately weighed amount of the ingredient equal to the amount present in a unit dose of the product into a 250-milliliter (mL) beaker; (2) if wetting is desired, add not more than 5 mL of alcohol (neutralized to an apparent pH of 3.5), and mix to wet the sample thoroughly; (3) add 70 mL of water, and mix on a magnetic stirrer at 300±30 r.p.m. for 1 minute (4) analyze the acid neutralizing capacity of the sample according to the aforementioned United States Pharmacopeia procedure and calculate the percent contribution of the acid neutralizing ingredient in the total product as follows:

Percent contribution=(Total mEq. Acid Neutralizing Ingredient×100)/(Total mEq. Acid Neutralizing Product)

There are a wide variety of available acid neutralizing/reducing products, most of which are in solid form, such as pills and tablets. Additionally, there are liquid products which are generally believed to be more effective than those in solid form, however they are better suited for a hospital setting rather than as an over-the-counter product.

Many pharmaceutical grade solid acid neutralizing agents known in the art are designed to be chewed in order to increase the overall surface area of the drug relative to the gastric acid in the patient's stomach or esophagus. In solid dosage products, however, a relatively large amount of active ingredient is required to provide optimal acid neutralizing effectiveness. Such a large amount of common acid neutralizing material, such as metal salts, leads to an unpleasant taste and mouth feel due to the chalkiness, grittiness, and dryness commonly associated with these materials. Accordingly, there have been several attempts at developing food products that include acid neutralizing materials in order to provide a better tasting alternative to traditional tablets or pills.

U.S. Pat. No. 5,665,376 describes a method for treating or reducing excess stomach acidity by ingesting a predetermined dose of flour or meal made from dried fava beans wherein the flour or meal may be incorporated into an edible food product such as a wafer, cookie, biscuit or drink mix. The dried fava bean material is used as the active antacid ingredient. Because fava bean flour is has a generally unpleasant taste, it may be combined with various sweeteners and other ingredients to form a wafer or cookie. In a preferred embodiment, fava bean flour may be used in a cookie as its only flour, in combination with shortening, sweetener and other common food ingredients.

U.S. Pat. No. 5,762,962 describes a pharmaceutical antacid composition for immediate and long lasting relief from the symptoms of acid indigestion. The composition may comprise a dual or tripartite combination of the actives calcium carbonate, calcium or magnesium citrate and/or calcium phosphate. The patent indicates that in addition to the antacid benefits, the composition may also serve as a dietary supplement of calcium. Further, the patent discloses that the antacid composition may be delivered in a liquid or solid dosage form, such as a tablet. Inactive ingredients such as starch, cellulose, corn syrup, sugars, sugar alcohols, assorted dyes, flavors and the like may included in the liquid or solid delivery dosage for improved taste and texture.

U.S. Pat. No. 6,174,542 describes a dietary supplement and food product for treating, preventing, or alleviating symptoms associated with premenstrual syndrome. The essential ingredients include kava kava and/or St. John's wort, cayenne, ginger, ginseng, chickweed and/or buchu, wild yam, vintage and mineral supplements. Food products incorporating these ingredients may include a shake, juice, hard candy, soft candy, gum, granola bars, cookies, brownies, etc.

U.S. Patent Application Publication Nos. 2007/0196496 and 2007/0292517 describe a variety of edible delivery systems for functional or medicinal ingredients. In one such example, disclosed is a delivery system for an antacid wherein the antacid is in a gel form and may be used as a confectionary filling or coating for baked goods such as wafers or cookies. Described is the use of a functional composition as part of a layer between two wafers, or a jelly layer on the top of a cookie or sponge cake wherein the product may be further coated with chocolate or other flavored coating. Alternatively, the antacid gel may be used to fill a doughnut-type baked good.

U.S. Patent Application Publication No. 2007/0292534 describes an antacid and breath freshening composition. The composition includes an effective amount of antacid such as sodium bicarbonate, sodium carbonate, potassium containing ingredients, aluminum containing ingredients, and calcium carbonate. The oral composition disclosed may be in the form of a soft chew, compressed tablet, or other orally dissolvable product. The composition may include carbohydrates, milk, flavoring, emulsifiers, and coloring. Additionally, the orally dissolvable product may include sweeteners to improve taste.

U.S. Patent Application Publication No. 2009/0130232 describes a composition and method for treating oral inflammation and ulceration. The anti-ulceration composition includes an antacid which may be aluminum carbonate, aluminum hydroxide, aluminum phosphate, bicarbonates, and bismuth containing compounds. In one embodiment, the composition comprises an anesthetic composition, an antifungal composition, a steroid composition, an antihistamine composition, and an antacid composition, wherein the composition is substantially free of alcohol.

Accordingly, while there are a variety of edible products containing acid neutralizing ingredients, there is a need in the prior art for such a product with improved taste, texture, mouth feel, and digestibility while also providing nutritional value as a dietary supplement. Specifically, there is a need in the art for an acid neutralizing product that is delivered to the patient in the form of a cookie.

### SUMMARY OF THE INVENTION

The subject invention is directed toward edible compositions that are effective for treating, preventing, and/or alleviating common symptoms associated with heartburn, acid reflux, and indigestion. In various embodiments, the edible composition of the present invention comprises: sweetener, flour, fat, leavening agents, and an effective amount of acid neutralizing material. Additionally, further embodiments of the present invention may include flavoring and/or spices. In addition to the various edible compositions themselves, the present invention concerns methods of preparing the compositions as well as methods of the reduction of stomach acidity by ingesting the edible compositions disclosed. Finally, certain embodiments of the present invention have the additional benefit of providing dietary supplementation of minerals with beneficial characteristics such as calcium.

Accordingly, it is a principal object of the present invention to provide an edible composition that is effective for reducing stomach acid while also providing improved taste, texture, mouth feel, and digestibility. It is a further object of the present invention to provide an alternative acid neutralizing product to many of the common tablets, pills, and liquids which are generally unappealing due to their grittiness, dryness, and unpleasant taste. It is a further object of the present invention for the edible composition herein disclosed to provide mineral and nutritional supplements to the consumer. These and other objectives of the various embodiments of the present invention will become readily apparent in the following specification and appended claims.

### DETAILED DESCRIPTION OF THE INVENTION

Disclosed herein are various embodiments of edible compositions suitable for reducing stomach acid. Further, the present invention is directed toward methods of preparing edible compositions and methods of reducing stomach acidity by ingesting the edible compositions described herein. Some embodiments of the present invention comprise a baked good, such as a cookie, containing an effective amount of acid neutralizing material. Further embodiments of the present invention comprise a baked good, such as a cookie, containing an effective amount of a calcium containing ingredient as a dietary supplement. For purposes of this disclosure, it should be understood that the term “cookie” shall not be construed as limiting, but rather is used in an exemplary fashion to describe the size and shape of one embodiment of the composition of the present invention. In at least one embodiment, the composition of the present invention may contain the following ingredients: sweetener, flour or starch, fat, a separating agent, a leavening agent, water, flavoring and/or spices, and an effective amount of acid neutralizing material.

### Ingredients

In some embodiments, the sweetener may be sugar, an artificial sweetener, or combinations thereof. Such sweeteners include, but are not limited to, table sugar (i.e. common white granulated sugar), brown sugar, sugar syrups, honey, corn syrup, high fructose corn syrup, hydrogenated glucose syrups, turbinado (i.e., sugar cane extract), xylose, ribose, sucrose, mannose, galactose, fructose, dextrose, maltose, polydextrose, aspartame (e.g., NUTRASWEET®), saccharin-based sweeteners (e.g., SWEET′N LOW®), a combination of aspartame, dextrose, and maltodextrin (e.g., EQUAL®), sucralose, sugar-alcohols such as glycol, glycerol, erythritol, threitol, arabitol, xylitol, ribiol, mannitol, sorbitol, dulcitol, and/or iditol, stevia, acesulfame potassium (also known as acesulfame-k or ace-k) and combinations thereof. Further still, such sweeteners may be sugar/sugar syrup combinations including, but not limited to, combinations of isomalt syrup, high fructose corn syrup, corn syrup and maltitol syrup, such as isomalt syrup and high fructose corn syrup, corn syrup and high fructose corn syrup, and maltitol syrup and high fructose corn syrup. In one embodiment, the total sweetener content is typically in the range of about 5% to about 40% by weight (henceforth, proportions of components given as “% by weight” shall mean the percentages by weight of raw ingredients prior to cooking or baking). In a further embodiment, the total sweetener content is in the range of about 20% to 30% by weight. In some embodiments, however, where some artificial sweeteners are used, the quantity of sweetener can be as low as 0.1% by weight and still provide sufficient sweetening.

In some embodiments, the flour or starch may include, but is not limited to, bread flour, whole wheat flour, buckwheat flour, cassaya flour, corn flour, oatmeal flour, rice flour, rye flour, tapioca flour, and combinations thereof. Further, suitable flours or starches may include cereal grains and meals or flours obtained from grains such as corn, oats, wheat, milo, barley, rice as a well as whet feed flour, wheat middlings, mixed feed, wheat shorts, wheat red dog, oat groats, hominy feed and other such materials. Other types of flours meals and starches readily known in the art may be selected depending on the desired taste, texture, and cooking requirements. In one embodiment, the total flour or starch content is typically in the range of about 20% to about 50% by weight. In a further embodiment, the total flour content is in the range of about 30% to 40% by weight.

In some embodiments, the fat may include various known solid or liquid shortenings or oils. Such fats include, but are not limited to, canola oil, rapeseed oil, corn oil, peanut oil, olive oil, palm oil, soybean oil, sunflower oil, pumpkin seed oil, safflower oil, grape seed oil, sesame oil, argan oil, rice bran oil, mustard oil, almond oil, walnut oil, diacylglycerol (DAG) oil, cottonseed oil, vegetable oil (i.e., various combinations of palm, corn, soybean, and sunflower oil), lard, processed vegetable shortening (i.e., Crisco®), butter, butter oil, clarified butter, margarine, Olestra (Olean®) or other non-caloric edible fats, mineral oils, and combinations thereof. Other types of shortenings and oils readily known in the art may be selected depending on the desired cooking and flavor requirements. In one embodiment, the total fat content is typically in the range of about 5% to about 25% by weight. In a further embodiment, the total fat content is in the range of about 5% to 10% by weight.

In some embodiments, the flavoring and/or spices may include a variety of known natural and artificial ingredients from various sources such as plants, leaves, flowers, fruits, and nuts. For example, such flavorings or spices may include, but are not limited to, vanilla extract, chocolate, peanut butter, apple butter, caramel, coco, cinnamon, nutmeg, salt, fruit oils (e.g., oils from the orange, apple, banana, strawberry, raspberry, blueberry, blackberry, date, fig, coconut), and combinations thereof. In one embodiment, the total flavoring and/or spice content is typically in the range of about 2% to about 10% by weight. In a further embodiment, the total flavoring content is in the range of about 3% to 7% by weight.

There are a wide variety of acid neutralizing materials that may be suitable for use in the present invention. Such materials may include, but are not limited to, aluminum containing ingredients such as alumina, aluminum carbonate, aluminum hydroxide, aluminum hydroxide-hexitol stabilized polymer, aluminum hydroxide-magnesium carbonate, aluminum hydroxide-magnesium trisilicate, aluminum-hydroxide sucrose, and aluminum phosphate, dihydroxyaluminum aminoacetate, dihydroxyaluminum; bentonite; bismuth containing ingredients such as bismuth aluminate, bismuth carbonate, bismuth subcarbonate, bismuth subgallate, bismuth subnitrate and bismuth subsalicylate; calcium carbonate; calcium phosphate; citric acid and salts thereof such as potassium citrate and sodium citrate; sodium carbonate; glycine; hydrotalcite; magnesium containing ingredients such as magnesia, magnesium alginate, magnesium carbonate, magnesium glycinate, magnesium hydroxide, magnesium oxide, magnesium phosphate, magnesium aluminum silicate and magnesium trisilicate; magaldrate; milk solids; potassium containing ingredients such as potassium bicarbonate and potassium phosphate; sodium bicarbonate; sodium polyhydroxy aluminium mono carbonate hexitol complex; sucralfate (a basic aluminium salt of sucrose octasulphate); tartaric acid and salts thereof such as sodium tartrate and sodium potassium tartrate; tricalcium phosphate and zeolite; magnesium aluminosilicates, magnesium trisilicate, hydrotalcite, and combinations thereof. In one embodiment, the total acid neutralizing content is typically in the range of about 2% to about 15% by weight. In a further embodiment, the total acid neutralizing content is in the range of about 5% to 10% by weight. It should be understood, however, that when selecting one or more acid neutralizing ingredients for use in the composition of the present invention, it is desirable to select a dosage that will provide optimal acidity buffering in accordance with the above mentioned United States Pharmacopoeia standards.

Accordingly, the proportion by weight of acid neutralizing material to be included in each serving of the product of the present invention shall be determined in accordance with the acid neutralizing capacity of the ingredient. As previously mentioned, in order to meet the United States Pharmacopoeia standards for effective acid neutralization, it is desirable for the acid neutralizing ingredient (i.e. a single effective ingredient or combination of effective ingredients), when added to 10 mL. of 0.5 N HCL (5 mEq), to produce a pH of 3.5 or greater after 10 minutes of stirring.

It should also be understood that the calcium-containing acid neutralizing ingredients identified above will also be effective for dietary calcium supplementation. Such ingredients will deliver calcium to the subject ingesting the composition of the present invention whereby the calcium may be helpful in treating and preventing a wide variety of conditions related to calcium deficiency. Additional benefits of calcium supplementation will be readily apparent to those knowledgeable in the art. In addition to calcium carbonate and calcium phosphate, other suitable calcium containing ingredients may include, but are not limited to, coral calcium (a combination of calcium carbonate and trace minerals), calcium citrate, calcium lactate, calcium chelates (i.e. synthetic calcium compounds, with calcium bound to an organic molecule, such as malate, aspartate, or fumarate), microcrystalline hydroxyapatite, and combinations thereof.

In some embodiments, the present invention includes one or more leavening agents to provide optimal baking, resulting in a cookie with a stable shape and size and desirable texture. Such leavening agents may include, but are not limited to, sodium bicarbonate, phosphates and combinations thereof. Additionally, a suitable leavening agent may also comprise baking powder, which comprises a mixture of at least one bicarbonate (e.g. sodium bicarbonate) or carbonate salt, at least one acidifying agent and at least one separating agent, and combinations thereof. In some embodiments, the leavening agent is a baking powder comprising sodium bicarbonate, sodium diphosphate or disodium dihydrogen pyrophosphate, and a starch. In addition to the leavening agent, some embodiments of the present invention may also include a separating agent such as, for example, sodium bicarbonate (e.g. baking soda), corn starch, or the like. Further, in some embodiments, it may be useful to combine sodium bicarbonate (e.g. baking soda) with baking powder (as described above) as desired to promote optimal leavening and separation for improved texture and mouth feel. In one embodiment, the total leavening/separating agent content is typically in the range of about 0.1% to about 5% by weight. In a further embodiment, the total leavening/separating agent content is in the range of about 0.2% to 1% by weight.

In some embodiments, the composition will have a sufficient amount of water such that it has a moisture content in the range of about 2% to about 30% by weight. In a further embodiment, the total moisture is in the range of about 8% to 20% by weight.

In some embodiments, in addition to the above ingredients, the present invention may include one or more flavoring additives which can either be incorporated in the cookie mix, used as a coating or topping thereon, or both. Such flavoring additives may include, but are not limited to, fruits such as apples, oranges, strawberries, raspberries, raisins, dates, cranberries, peaches, mangos; whole or partial nuts such as peanuts, cashews, almonds, brazils, macadamia nuts; confectionaries such as fudge, candies, candy coated chocolates, and combinations thereof.

### Preparation

In one embodiment, the present invention is prepared according to the following method:

First, the sweetener and fat is combined in a mixer until smooth and absent any lumps or chunks. In one embodiment, a planetary mixer may be used, however it is not necessary to use an electric mixer. Next, the flour, acid neutralizing ingredient and leavening/separating agents (“dry” ingredients) are combined until thoroughly and evenly mixed. Next, the “dry” ingredients are combined with the sweetener/fat mixture and again mixed thoroughly. Next, the water and any desired flavorings or spices are added to the mixture and the resulting dough is mixed thoroughly until it stiffens and becomes a heavy dough. The heavy dough is then allowed to stand at room temperature (about 72 degrees Fahrenheit) for approximately thirty (30) minutes. Next, the dough is rolled out and is then divided into individual servings. Finally, the dough is placed on a baking sheet, whereby the baking sheet placed in a convection oven and the dough is baked until thoroughly cooked. Of course the foregoing is merely an exemplary method of preparing one embodiment of the present invention; modifications to this embodiment of the method will be apparent to those skilled in the art depending on the desired taste, texture, size, and shape of the resulting product.

As mentioned previously, for the purposes of this disclosure, the term “cookie” shall not be construed as limiting. Rather, the above disclosed composition may be equally well suited in other forms such as, for exemplary purposes only, a granola bar, energy bar, brownie, cake, or wafer.

### EXAMPLES

The following is an example of one method of preparing the composition of the present invention according to the above procedure:

First, 250 grams of table sugar is creamed with 84 grams of vegetable shortening in a planetary mixture until the mixture is smooth and absent lumps. Next, in a separate container, 360 grams of bread flour is combined with 84 grams of calcium carbonate (i.e. the acid neutralizing/calcium supplementation material), 3 grams of baking powder, and 3 grams of sodium bicarbonate (“dry” ingredients). Next, the “dry” ingredients are combined with the sugar/shortening mixture in the planetary mixer. Next, 2 fl oz. of vanilla extract and 3.5. fl oz. of water are added to the planetary mixer while it is running. The resulting mixture is then mixed until it stiffens and becomes a heavy dough. Next, the dough is allowed to stand for approximately 30 minutes. The dough is then rolled out to a thickness of about 1/16″ and divided into sections of approximately 10 grams each. Finally, the product is baked in a convection oven at 325 degrees Fahrenheit for approximately 12 minutes. The product is allowed to cool and thereafter it is ready to be consumed and/or packaged for shipment.

In accordance with the above example, Table 1 below describes the composition by weight of each ingredient therein:

TABLE 1

| Ingredient | % by Weight |
|---|---|
| Sugar | 26.41% |
| Vegetable Shortening | 8.87% |
| Bread Flour | 38.03% |
| Calcium Carbonate | 8.87% |
| Baking Powder | 0.32% |
| Sodium Bicarbonate | 0.32% |
| Pure Vanilla Extract | 6.25% |
| Water | 10.93% |
| Total | 100% |

As shown, this embodiment of the present invention contains approximately 8.87% by weight of effective acid neutralizing material (e.g. calcium carbonate). In one embodiment, a single serving of the present invention may weigh about 10 grams and therefore will contain about 887 mg (0.887 grams) of acid neutralizing material. This amount of calcium carbonate has an acid neutralizing capacity above that which is mandated by the United States Pharmacopeia standard (i.e. when added to 10 mL of 0.5 N HCL (5 mEq), produces a pH of 3.5 or greater after 10 minutes of stirring.) More specifically, testing has shown that this embodiment has an acid neutralizing capacity that is capable of producing a pH of 5.02 after 10 minutes of stirring when added to 10 mL of 0.5 N HCL. Accordingly, to reduce stomach acid, an individual consumes one or more servings of the present invention whereby the acid neutralizing material is introduced into the stomach and thereafter buffers stomach acid.

Table 2 below describes the composition by weight of another example of one embodiment of the present invention, which includes salt and an artificial sweetener:

TABLE 2

| Ingredient | % by Weight |
|---|---|
| Sugar | 15.65% |
| Sucralose | 0.0034% |
| Shortening | 10.43% |
| Bread Flour | 44.72% |
| Calcium Carbonate | 8.45% |
| Salt | 0.37% |
| Baking Powder | 0.25% |
| Sodium Bicarbonate | 0.25% |
| Pure Vanilla Extract | 7.45% |
| Water | 12.42% |
| Total | 100% |

As shown, this embodiment of the present invention contains approximately 8.45% by weight of effective acid neutralizing material (e.g. calcium carbonate). In one embodiment, a single serving of the present invention may weigh about 8.88 grams (precooked) and therefore will contain about 750 mg (0.750 grams) of acid neutralizing material.

It should be understood that the present invention is not limited to compositions only having a quantity of acid neutralizing material that, when added to 10 mL of 0.5 N HCL (5 mEq), produce a pH of 3.5 or greater after 10 minutes of stirring. For example, it is estimated by the inventor that some embodiments of the present composition having a quantity of acid neutralizing material that, when added to 10 mL of 0.25 N HCL (2.5 mEq), produce a pH of 3.5 or greater after 10 minutes of stirring will also be effective as an antacid to some extent. Further still, some embodiments of the present invention may have a quantity of acid neutralizing material that produces a pH of 3.5 or greater after 10 minutes of stirring when added to 10 ml of 0.75 N HCL (7.5 mEq).

In the foregoing description, the present invention has been described with reference to specific exemplary embodiments thereof. It will be apparent to those skilled in the art that a person understanding this invention may conceive of changes or other embodiments or variations, which utilize the principles of this invention without departing from the broader spirit and scope of the invention. The specification and drawings are, therefore, to be regarded in an illustrative rather than a restrictive sense. Accordingly, it is not intended that the invention be limited except as may be necessary in view of the appended claims.

I claim:

1. An edible composition for reducing stomach acid comprising:
a. between about 0.1% and about 40% by weight of sweetener;
b. between about 20% and about 50% by weight of flour;
c. between about 5% and about 25% by weight of fat;
d. between about 0.1% and about 5% by weight of leavening agent;
e. between about 2% and about 30% by weight of water; and
f. an effective quantity of acid neutralizing material.

2. The composition according to claim 1 wherein said sweetener is selected from the group consisting of table sugar, brown sugar, sugar syrups, honey, corn syrup, high fructose corn syrup, hydrogenated glucose syrups, turbinado, xylose, ribose, sucrose, mannose, galactose, fructose, dextrose, maltose, polydextrose, aspartame, saccharin, a combination of aspartame, dextrose, and maltodextrin, sucralose, glycol, glycerol, erythritol, threitol, arabitol, xylitol, ribiol, mannitol, sorbitol, dulcitol, iditol, isomalt syrup, high fructose corn syrup, corn syrup and maltitol syrup stevia, acesulfame potassium, and combinations thereof.

3. The composition according to claim 1 wherein said flour is selected from the group consisting of: bread flour, whole wheat flour, buckwheat flour, cassaya flour, corn flour, oatmeal flour, rice flour, rye flour, tapioca flour, cornmeal, milo meal, barley meal, rice meal, and combinations thereof.

4. The composition according to claim 1 wherein said fat is selected from the group consisting of canola oil, rapeseed oil, corn oil, peanut oil, olive oil, palm oil, soybean oil, sunflower oil, pumpkin seed oil, safflower oil, grape seed oil, sesame oil, argan oil, rice bran oil, mustard oil, almond oil, walnut oil, diacylglycerol oil, cottonseed oil, vegetable oil, lard, processed vegetable shortening, butter, butter oil, clarified butter, margarine, Olestra (Olean®) or other non-caloric edible fats, mineral oils, and combinations thereof.

5. The composition according to claim 1 wherein said leavening agent is selected from the group consisting of sodium bicarbonate, sodium diphosphate, disodium dihydrogen pyrophosphate, baking powder, and combinations thereof.

6. The composition according to claim 1 wherein said acid neutralizing material is selected from the group consisting of: alumina, aluminum carbonate, aluminum hydroxide, aluminum hydroxide-hexitol stabilized polymer, aluminum hydroxide-magnesium carbonate, aluminum hydroxide-magnesium trisilicate, aluminum-hydroxide sucrose, aluminum phosphate, dihydroxyaluminum aminoacetate, dihydroxyaluminum; bentonite, bismuth aluminate, bismuth carbonate, bismuth subcarbonate, bismuth subgallate, bismuth subnitrate, bismuth subsalicylate, calcium carbonate, calcium phosphate, potassium citrate, sodium citrate, sodium carbonate, glycine, hydrotalcite, magnesia, magnesium alginate, magnesium carbonate, magnesium glycinate, magnesium hydroxide, magnesium oxide, magnesium phosphate, magnesium aluminum silicate, magnesium trisilicate; magaldrate; milk solids, potassium bicarbonate, potassium phosphate, sodium bicarbonate, sodium polyhydroxy aluminium mono carbonate hexitol complex, sucralfate, tartaric acid, sodium tartrate and sodium potassium tartrate, tricalcium phosphate, zeolite; magnesium aluminosilicates, magnesium trisilicate, hydrotalcite, and combinations thereof.

7. The composition according to claim 6 wherein said effective quantity of said acid neutralizing material is the quantity that when added to 10 mL of 0.5 N HCL (5 mEq), produces a pH of 3.5 or greater after 10 minutes of stirring.

8. The composition according to claim 6 wherein said effective quantity of said acid neutralizing material is the quantity that when added to 10 mL of 0.25 N HCL (2.5 mEq), produces a pH of 3.5 or greater after 10 minutes of stirring.

9. The composition according to claim 6 wherein said effective quantity of said acid neutralizing material is the quantity that when added to 10 mL of 0.75 N HCL (7.5 mEq), produces a pH of 3.5 or greater after 10 minutes of stirring.

10. The composition according to claim 1 further comprising between about 2% and about 10% by weight of flavoring wherein said flavoring is selected from the group consisting of: vanilla extract, chocolate, peanut butter, apple butter, caramel, coco, cinnamon, nutmeg, salt, fruit oils, and combinations thereof.

11. The composition according to claim 1, wherein said composition comprises between about 20% and about 30% by weight of sweetener.

12. The composition according to claim 1, wherein said composition comprises between about 30% and about 40% by weight of flour.

13. The composition according to claim 1, wherein said composition comprises between about 5% and about 10% by weight of fat.

14. The composition according to claim 1, wherein said composition comprises between about 0.2% and about 1% by weight of leavening agent.

15. The composition according to claim 1, wherein said composition comprises between about 8% and about 20% by weight of water.

16. The composition according to claim 1, wherein said composition comprises between about 23% and about 28% by weight of sweetener.

17. The composition according to claim 1, wherein said composition comprises between about 35% and about 39% by weight of flour.

18. The composition according to claim 1, wherein said composition comprises between about 8% and about 10% by weight of fat.

19. The composition according to claim 1, wherein said composition comprises between about 0.4% and about 0.8% by weight of leavening agent.

20. The composition according to claim 1, wherein said composition comprises between about 9% and about 12% by weight of water.

21. The composition according to claim 1, wherein said composition comprises about 26% by weight of sweetener.

22. The composition according to claim 1, wherein said composition comprises about 38% by weight of flour.

23. The composition according to claim 1, wherein said composition comprises about 9% by weight of fat.

24. The composition according to claim 1, wherein said composition comprises about 0.6% by weight of leavening agent.

25. The composition according to claim 1, wherein said composition comprises about 11% by weight of water.

26. A method for reducing stomach acid by ingesting an edible composition, wherein said composition comprises:
a. between about 0.1% and about 40% by weight of sweetener;
b. between about 20% and about 50% by weight of flour;
c. between about 5% and about 25% by weight of fat;
d. between about 0.1% and about 5% by weight of leavening agent;
e. between about 2% and about 30% by weight of water; and
f. an effective quantity of acid neutralizing material.

27. The composition according to claim 1 wherein said sweetener is selected from the group consisting of: table sugar, brown sugar, sugar syrups, honey, corn syrup, high fructose corn syrup, hydrogenated glucose syrups, turbinado, xylose, ribose, sucrose, mannose, galactose, fructose, dextrose, maltose, polydextrose, aspartame, saccharin, a combination of aspartame, dextrose, and maltodextrin, sucralose, glycol, glycerol, erythritol, threitol, arabitol, xylitol, ribiol, mannitol, sorbitol, dulcitol, iditol, isomalt syrup, high fructose corn syrup, corn syrup and maltitol syrup stevia, acesulfame potassium, and combinations thereof.

28. The method according to claim 26 wherein said flour is selected from the group consisting of: bread flour, whole wheat flour, buckwheat flour, cassaya flour, corn flour, oatmeal flour, rice flour, rye flour, tapioca flour, cornmeal, milo meal, barley meal, rice meal, and combinations thereof.

29. The method according to claim 26 wherein said fat is selected from the group consisting of: canola oil, rapeseed oil, corn oil, peanut oil, olive oil, palm oil, soybean oil, sunflower oil, pumpkin seed oil, safflower oil, grape seed oil, sesame oil, argan oil, rice bran oil, mustard oil, almond oil, walnut oil, diacylglycerol oil, cottonseed oil, vegetable oil, lard, processed vegetable shortening, butter, butter oil, clarified butter, margarine, Olestra (Olean®) or other non-caloric edible fats, mineral oils, and combinations thereof.

30. The method according to claim 26 wherein said leavening agent is selected from the group consisting of: sodium bicarbonate, sodium diphosphate, disodium dihydrogen pyrophosphate, baking powder, and combinations thereof.

31. The method according to claim 26 wherein said acid neutralizing material is selected from the group consisting of: alumina, aluminum carbonate, aluminum hydroxide, aluminum hydroxide-hexitol stabilized polymer, aluminum hydroxide-magnesium carbonate, aluminum hydroxide-magnesium trisilicate, aluminum-hydroxide sucrose, aluminum phosphate, dihydroxyaluminum aminoacetate, dihydroxyaluminum; bentonite, bismuth aluminate, bismuth carbonate, bismuth subcarbonate, bismuth subgallate, bismuth subnitrate, bismuth subsalicylate, calcium carbonate, calcium phosphate, potassium citrate, sodium citrate, sodium carbonate, glycine, hydrotalcite, magnesia, magnesium alginate, magnesium carbonate, magnesium glycinate, magnesium hydroxide, magnesium oxide, magnesium phosphate, magnesium aluminum silicate, magnesium trisilicate; magaldrate; milk solids, potassium bicarbonate, potassium phosphate, sodium bicarbonate, sodium polyhydroxy aluminium mono carbonate hexitol complex, sucralfate, tartaric acid, sodium tartrate and sodium potassium tartrate, tricalcium phosphate, zeolite; magnesium aluminosilicates, magnesium trisilicate, hydrotalcite, and combinations thereof.

32. The method according to claim 31 wherein said effective quantity of said acid neutralizing material is the quantity that when added to 10 mL of 0.5 N HCL (5 mEq), produces a pH of 3.5 or greater after 10 minutes of stirring.

33. The method according to claim 31 wherein said effective quantity of said acid neutralizing material is the quantity that when added to 10 mL of 0.25 N HCL (2.5 mEq), produces a pH of 3.5 or greater after 10 minutes of stirring.

34. The method according to claim 31 wherein said effective quantity of said acid neutralizing material is the quantity that when added to 10 mL of 0.75 N HCL (7.5 mEq), produces a pH of 3.5 or greater after 10 minutes of stirring.

35. The method according to claim 26 further comprising between about 2% and about 10% by weight of flavoring wherein said flavoring is selected from the group consisting of: vanilla extract, chocolate, peanut butter, apple butter, caramel, coco, cinnamon, nutmeg, salt, fruit oils, and combinations thereof.

36. The method according to claim 26, wherein said composition comprises between about 20% and about 30% by weight of sweetener.

37. The method according to claim 26, wherein said composition comprises between about 30% and about 40% by weight of flour.

38. The method according to claim 26, wherein said composition comprises between about 5% and about 10% by weight of fat.

39. The method according to claim 26, wherein said composition comprises between about 0.2% and about 1% by weight of leavening agent.

40. The method according to claim 26, wherein said composition comprises between about 8% and about 20% by weight of water.

41. The method according to claim 26, wherein said composition comprises between about 23% and about 28% by weight of sweetener.

42. The method according to claim 26, wherein said composition comprises between about 35% and about 39% by weight of flour.

43. The method according to claim 26, wherein said composition comprises between about 8% and about 10% by weight of fat.

44. The method according to claim 26, wherein said composition comprises between about 0.4% and about 0.8% by weight of leavening agent.

45. The method according to claim 26, wherein said composition comprises between about 9% and about 12% by weight of water.

46. The method according to claim 26, wherein said composition comprises about 26% by weight of sweetener.

47. The method according to claim 26, wherein said composition comprises about 38% by weight of flour.

48. The method according to claim 26, wherein said composition comprises about 9% by weight of fat.

49. The method according to claim 26, wherein said composition comprises about 0.6% by weight of leavening agent.

50. The method according to claim 26, wherein said composition comprises about 11% by weight of water.

51. A method of preparing an edible composition for reducing stomach acid comprising:
a. combining sweetener and fat until smooth, resulting in a first mixture;
b. combining flour, an effective quantity of acid neutralizing material, and leavening agent until thoroughly and evenly mixed, resulting in a second mixture;
c. combining said first mixture and said second mixture, resulting in a third mixture;
d. adding water to said third mixture, resulting in a dough;
e. heating said dough until said dough is thoroughly cooked.

52. The method according to claim 51 further comprising adding flavoring to said third mixture.

53. The method according to claim 51 further comprising allowing said dough to stand prior to step e.

54. The method according to claim 51 further comprising the step of rolling said dough out to a substantially even thickness of 1/16″ prior to step (e).

55. The method according to claim 54 further comprising the step of dividing said dough into single servings prior to step (e).

56. The method according to claim 51 wherein said heating comprises baking said dough in a convection oven.

57. The method according to claim 56 wherein said convection oven is at a temperature of about 325 degrees Fahrenheit.

58. The method according to claim 51 where said edible composition comprises:
a. between about 0.1% and about 40% by weight of said sweetener;
b. between about 20% and about 50% by weight of said flour;
c. between about 5% and about 25% by weight of said fat;
d. between about 0.1% and about 5% by weight of said leavening agent;
e. between about 2% and about 30% by weight of said water; and
f. a quantity of acid neutralizing material wherein said quantity is the quantity that when added to 10 mL of 0.5 N HCL (5 mEq), produces a pH of 3.5 or greater after 10 minutes of stirring.

59. An edible composition comprising:
a. between about 0.1% and about 40% by weight of sweetener;
b. between about 20% and about 50% by weight of flour;
c. between about 5% and about 25% by weight of fat;
d. between about 0.1% and about 5% by weight of leavening agent;
e. between about 2% and about 30% by weight of water; and
f. between about 5% and 25% by weight of a calcium-containing ingredient.

60. The composition according to claim 59 wherein said sweetener is selected from the group consisting of table sugar, brown sugar, sugar syrups, honey, corn syrup, high fructose corn syrup, hydrogenated glucose syrups, turbinado, xylose, ribose, sucrose, mannose, galactose, fructose, dextrose, maltose, polydextrose, aspartame, saccharin, a combination of aspartame, dextrose, and maltodextrin, sucralose, glycol, glycerol, erythritol, threitol, arabitol, xylitol, ribiol, mannitol, sorbitol, dulcitol, iditol, isomalt syrup, high fructose corn syrup, corn syrup and maltitol syrup stevia, acesulfame potassium, and combinations thereof.

61. The composition according to claim 59 wherein said flour is selected from the group consisting of: bread flour, whole wheat flour, buckwheat flour, cassaya flour, corn flour, oatmeal flour, rice flour, rye flour, tapioca flour, cornmeal, milo meal, barley meal, rice meal, and combinations thereof.

62. The composition according to claim 59 wherein said fat is selected from the group consisting of: canola oil, rapeseed oil, corn oil, peanut oil, olive oil, palm oil, soybean oil, sunflower oil, pumpkin seed oil, safflower oil, grape seed oil, sesame oil, argan oil, rice bran oil, mustard oil, almond oil, walnut oil, diacylglycerol oil, cottonseed oil, vegetable oil, lard, processed vegetable shortening, butter, butter oil, clarified butter, margarine, Olestra (Olean®) or other non-caloric edible fats, mineral oils, and combinations thereof.

63. The composition according to claim 59 wherein said leavening agent is selected from the group consisting of: sodium bicarbonate, sodium diphosphate, disodium dihydrogen pyrophosphate, baking powder, and combinations thereof.

64. The composition according to claim 59 wherein said calcium containing ingredient is selected from the group consisting of: calcium carbonate, calcium phosphate, coral calcium, calcium citrate, calcium lactate, calcium chelates, and microcrystalline hydroxyapatite, and combinations thereof.
